(19) United States
(12) Patent Application Publication
(10) Pub. No.: US 2025/0216959 A1
(43) Pub. Date: Jul. 3, 2025

(54) OPERATING APPARATUS AND INPUT APPARATUS FOR ULTRASOUND IMAGING SYSTEM, AND ULTRASOUND IMAGING SYSTEM

(71) Applicant: GE Precision Healthcare LLC, Waukesha, WI (US)

(72) Inventors: Zhihua Jia, Jiangsu (CN); Xiaoyun Ge, Jiangsu (CN); Kaixin Gu, Jiangsu (CN); Han Cheng, Jiangsu (CN); Gang Liu, Jiangsu (CN)

(21) Appl. No.: 18/990,703

(22) Filed: Dec. 20, 2024

(30) Foreign Application Priority Data
Dec. 29, 2023 (CN) 202311846397.1

(51) Int. Cl. G06F 3/0354

(52) U.S. Cl. CPC G06F 3/03549; G06F 3/03547

(57) ABSTRACT

An operating apparatus and an input apparatus for an ultrasound imaging system, and an ultrasound imaging system. The operating apparatus includes a plurality of operating surfaces, wherein at least one of the plurality of operating surfaces is mounted with a trackball assembly and at least one other of the plurality of operating surfaces is mounted with a touchpad assembly, and the operating apparatus can be flipped so that one of the trackball assembly and the touchpad assembly can be selected for controlling the ultrasound imaging system.

### CROSS-REFERENCE TO RELATED APPLICATION

This application claim priority to Chinese Patent Application No. 202311846397.1, which was file on Dec. 29, 2023 at the Chinese Patent Office. The entire contents of the above-listed application are incorporated by reference herein in their entirety.

### TECHNICAL FIELD

The present invention relates to the field of ultrasonic detection, and in particular to an operating apparatus and an input apparatus for an ultrasound imaging system, and an ultrasound imaging system.

### BACKGROUND

Ultrasound imaging is a medical imaging technique for imaging organs and soft tissues in the human body. Ultrasonic imaging uses real-time and non-invasive high-frequency sound waves to produce two-dimensional (2D) images, three-dimensional (3D) images, and/or four-dimensional (4D) images (i.e., real-time/continuous 3D images).

Ultrasound imaging systems may have different forms of input apparatuses, for example, trackballs in some ultrasound imaging systems, and touchpads in some ultrasound imaging systems. The trackball is a conventional input apparatus and can provide a user with a good feeling of use and feedback. As a new input apparatus, the touchpad is commonly used in smaller ultrasound imaging systems and can provide more functions and applications. However, in practical use, some more experienced or older operators prefer to use trackballs, whereas some younger operators prefer to use touchpads. For the same ultrasound imaging system, only one input apparatus is usually equipped, and it is impossible for different operators to select or change according to their preferences.

### SUMMARY

The present invention provides an operating apparatus and an input apparatus for an ultrasound imaging system, and an ultrasound imaging system.

Exemplary embodiments of the present invention provide an operating apparatus for an ultrasound imaging system, the operating apparatus comprising a plurality of operating surfaces, wherein at least one of the plurality of operating surfaces is mounted with a trackball assembly and at least one other of the plurality of operating surfaces is mounted with a touchpad assembly, and the operating apparatus can be flipped so that one of the trackball assembly and the touchpad assembly can be selected and then used for controlling the ultrasound imaging system.

Exemplary embodiments of the present invention further provide an input apparatus for an ultrasound imaging system, the input apparatus comprising an operating apparatus and an operation panel, wherein the operating apparatus comprises a plurality of operating surfaces, at least one of the plurality of operating surfaces is mounted with a trackball assembly and at least one other of the plurality of operating surfaces is mounted with a touchpad assembly, the operating apparatus can be flipped so that one of the trackball assembly and the touchpad assembly can be selected and then used for controlling the ultrasound imaging system, and the operation panel has a frame to which the operating apparatus can be mounted.

Exemplary embodiments of the present invention further provide an ultrasound imaging system comprising an input apparatus, the input apparatus comprising an operating apparatus and an operation panel, wherein the operating apparatus comprises a plurality of operating surfaces, at least one of the plurality of operating surfaces is mounted with a trackball assembly and at least one other of the plurality of operating surfaces is mounted with a touchpad assembly, the operating apparatus can be flipped so that one of the trackball assembly and the touchpad assembly can be selected and then used for controlling the ultrasound imaging system, and the operation panel has a frame to which the operating apparatus can be mounted.

Other features and aspects will become apparent from the following detailed description, drawings, and claims.

### BRIEF DESCRIPTION OF THE DRAWINGS

The present invention can be better understood by means of the description of the exemplary embodiments of the present invention in conjunction with the drawings, in which:

FIG. 1 is a schematic view of an ultrasound imaging system according to some embodiments of the present invention;

FIG. 2 is a schematic view of an operating apparatus according to some embodiments of the present invention from a first angle;

FIG. 3 is a schematic view of the operating apparatus according to some embodiments of the present invention from a second angle;

FIG. 4 is a schematic view of a user interface according to some embodiments of the present invention;

FIG. 5 is a schematic view of a user interface according to some other embodiments of the present invention;

FIG. 6 is a schematic view of a user interface according to still other embodiments of the present invention;

FIG. 7 is an exploded view of an input apparatus according to some embodiments of the present invention; and

FIG. 8 is an exploded view of an input apparatus according to some other embodiments of the present invention.

### DETAILED DESCRIPTION

Specific embodiments of the present invention will be described below. It should be noted that in the specific description of said embodiments, for the sake of brevity and conciseness, the present description cannot describe all of the features of the actual embodiments in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first” and “second” and similar terms used in the description and claims of the patent application of the present invention do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “connected” and similar words are not limited to physical or mechanical connections, and are not limited to direct or indirect connections.

The terms “scan” or “scanning” may also be used in the present disclosure to refer to acquiring data through the process of transmitting and receiving ultrasound signals. The terms “data” and “ultrasound data” may be used in the present disclosure to refer to one or a plurality of datasets acquired using an ultrasonic imaging system.

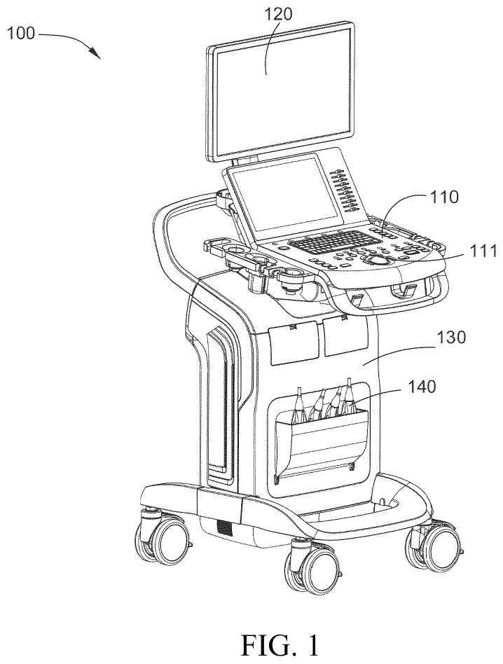

FIG. 1 shows an ultrasound imaging system 100 according to some embodiments of the present invention. The ultrasound imaging system 100 includes an input apparatus 110, a display apparatus 120, a host 130, and an ultrasound probe 140. Although FIG. 1 shows a desktop ultrasound imaging system, it should be understood by those skilled in the art that the ultrasound imaging system may also be a laptop computer, a handheld or portable system, or any other configuration.

The input apparatus 110 may include different types of input apparatuses that can communicate information to one or more processors of the ultrasound imaging system 100 from a user and/or at the direction of the user. The input apparatus may include a trackball or touchpad, a rotary encoder, a button, a keyboard, a mouse apparatus, a camera, a voice recognition microphone, and/or any other apparatuses capable of receiving user instructions.

The input apparatus 110 includes an operating apparatus 111. In some embodiments, the operating apparatus 111 may be a trackball apparatus. In some other embodiments, the operating apparatus 111 may also be a touchpad assembly. Furthermore, the input apparatus 110 further includes an operation panel including a frame for mounting the operating apparatus 111.

The host 130 may include a signal processing unit such as one or more processors in electronic communication with the ultrasound probe 140. The processor may control the ultrasound probe 140 to acquire ultrasound data, and is used for receiving acquired image information from the ultrasound probe, and processing and storing the image information. The processor is also in electronic communication with the display apparatus 120, and the processor may process the ultrasound data into an image(s) for display on the display apparatus 120. For the purpose of the present disclosure, the term “electronic communication” may be defined to include wired connection and wireless connection. According to some embodiments, the processor may include a central processing unit (CPU). According to other embodiments, the processor may include other electronic components capable of performing processing functions, for example, a digital signal processor (DSP), a field-programmable gate array (FPGA), a graphics processing unit (GPU), or any other type of processor. According to other embodiments, the processor may include a plurality of electronic components capable of performing processing functions. For example, the processor may include two or more electronic components selected from a list including the following electronic components: a central processing unit (CPU), a digital signal processor (DSP), a field-programmable gate array (FPGA), and a graphics processing unit (GPU).

The display apparatus 120 may be any apparatus capable of communicating visual information to the user. For example, the display apparatus 120 may include a liquid crystal display, a light-emitting diode display, and/or any suitable one or more displays. The display apparatus 120 may be a touch screen display that is configured to receive a user instruction via touch input. The display apparatus 120 may be operable to display information from one or more processors or memories of the ultrasound imaging system 100. The display apparatus includes a user interface that may display at least one ultrasound image, other marks or tools, etc.

The input apparatus 110, the display apparatus 120, and the host 130 may be a part of the body of the ultrasound imaging system 100. For example, the display apparatus, the input apparatus, and the host may be integrally provided in the frame, or, in some portable devices, they may be detachably connected to the frame as a part of the body of a portable ultrasound imaging apparatus.

The ultrasound probe 140 may include a probe connector, an ultrasound transducer, and a cable. The probe connector may be used to electrically connect to the body of the ultrasound imaging system 100. For example, one or more connector sockets are provided on the host 130, and the probe connector can be plugged into the socket to achieve an electrical connection. The ultrasonic transducer is used for transmitting and receiving ultrasonic signals, and includes a transducer housing and a piezoelectric element disposed in the housing. The ultrasound transducer is electrically connected to the probe connector by means of the cable. On the one hand, the cable can supply power to the ultrasound transducer, and on the other hand, it is used for data transmission during operation of the ultrasound imaging system 100.

According to various embodiments, the ultrasound probe may be any type of probe, including a linear probe, a curved array probe, a 1.25D array probe, a 1.5D array probe, a 1.75D array probe, or a 2D array probe. According to other embodiments, the probe may also be a mechanical probe, for example, a mechanical 4D probe or a hybrid probe. The probe may be used to acquire 4D ultrasound data, and the 4D ultrasound data includes information about how a volume changes over time. Each volume may include a plurality of 2D images or slices. According to some embodiments, the ultrasound probe may include an electronic circuit so as to perform all or part of the work of transmit beamforming and/or receive beamforming.

To facilitate an operator to select a trackball or a touchpad according to his own preference to operate the ultrasound imaging system, the present invention proposes an operating apparatus wherein a trackball and a touchpad are combined into one. The trackball and the touchpad are integrated on the same overall structure, for example, placed on different operating surfaces of the structure, so that the operating apparatus can be operated (for example, flipped) by the operator to select the trackball or touchpad for display or exposure on the operating apparatus, thereby enabling a user to operate the trackball or touchpad to operate or control the ultrasound imaging system. Such a selection or operation method is simple and easy to operate without replacement or operation performed by a professional engineer, and the operator can select an appropriate operation panel according to his own preference.

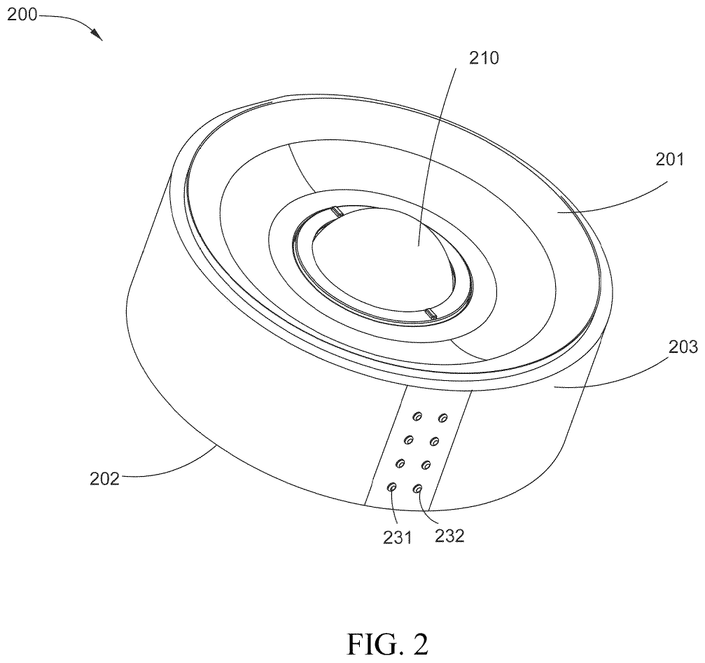

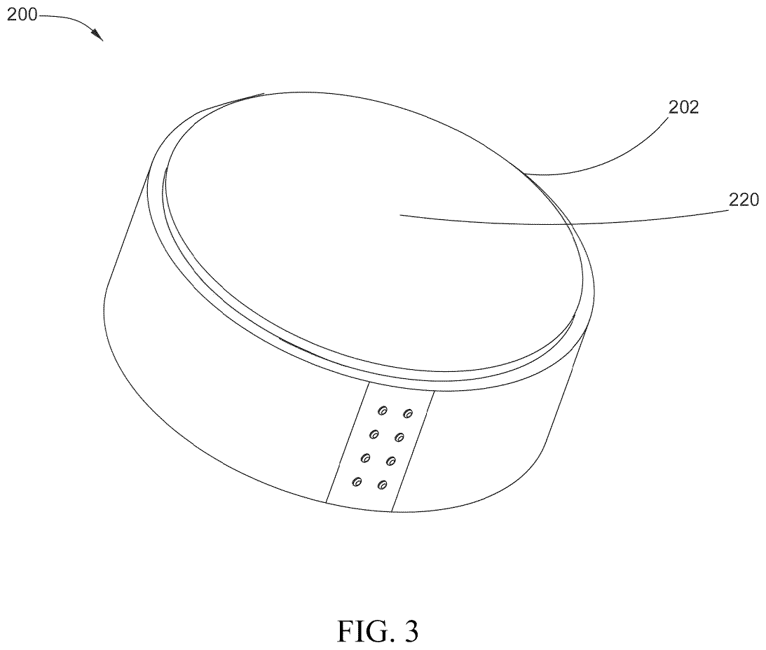

FIG. 2 shows a schematic view of an operating apparatus according to some embodiments of the present invention from a first angle, and FIG. 3 shows a schematic view of the operating apparatus according to some embodiments of the present invention from a second angle. As shown in FIGS. 2-3, the operating apparatus 200 includes a plurality of operating surfaces. At least one of the plurality of operating surfaces is mounted with a trackball assembly 210, and at least one other of the plurality of operating surfaces is mounted with a touchpad assembly 220. The operating apparatus 200 can be flipped so that one of the trackball assembly 210 and the touchpad assembly 220 can be selected and then used for controlling the ultrasound imaging system.

In the trackball assembly, the rotation direction and/or speed of a ball rotated by a user are/is detected by a pair of encoders corresponding to an X axis and a Y axis that are orthogonally configured, and a corresponding control signal is generated according to the detected rotation direction and/or speed. In the touchpad assembly, the trajectory and/or speed of a user's finger on a touchpad is detected by a touchpad sensor or the like, thereby detecting the movement and position of the finger, and then generating a corresponding control signal.

Specifically, the trackball assembly 210 includes a cover plate and a ball, the cover plate has a through opening portion at the center thereof, and the ball is accommodated in the operating apparatus in a freely rotatable state, with a part of the ball exposed from the opening portion. In some embodiments, the operating apparatus is internally provided with a detector to detect input information caused by the rotation of the ball. For example, a pair of encoders corresponding to an X axis and a Y axis (X/Y encoder for short hereinafter) may be configured in the accommodating portion. The rotation direction and/or speed of the ball rotated by the user can be detected by the X/Y encoder, and a corresponding cursor or pointer may be displayed on a display screen according to the detected rotation direction and/or speed. The detector may be electrically connected to an external device. For example, power may be obtained from the outside. As another example, detection information may be transmitted to a corresponding device.

Specifically, the touchpad assembly 220 includes a touchpad and a circuit board (not shown in the figures) accommodated in the operating apparatus, and the circuit board is provided with a touchpad sensor, so as to perform corresponding control by detecting an operation of the user or the operator. The touchpad sensor may be electrically connected to an external device. For example, power may be obtained from the outside. As another example, detection information may be transmitted to a corresponding device.

The plurality of operating surfaces include a first surface 201 and a second surface 202 that are disposed opposite to each other, and a side surface 203 connected between the first surface 201 and the second surface 202. In some embodiments, the trackball assembly 210 is mounted on the first surface 201, and the touchpad assembly 220 is mounted on the second surface 202. That is, the trackball assembly 210 and the touchpad assembly 220 are mounted on two opposite surfaces of the operating apparatus. Specifically, the cover plate in the trackball assembly may form a first surface of the operating apparatus, with a part of the ball exposed from the first surface, whereas the touchpad in the touchpad assembly is mounted on the second surface 202, or the touchpad may form the second surface 202 of the operating apparatus.

In some other embodiments, the trackball assembly 210 and the touchpad assembly 220 may be mounted on any two adjacent surfaces of the operating apparatus, for example, the first surface 201 and the side surface 203, respectively.

Of course, a plurality of trackball assemblies 210 or a plurality of touchpad assemblies 220 may also be provided on the plurality of surfaces of the operating apparatus. For example, one trackball assembly 210 may be provided on the first surface of the operating apparatus, and a plurality of touchpad assemblies 220 may be provided on the second surface and/or the side surface of the operating apparatus, respectively. Alternatively, one touchpad assembly 220 may be provided on the first surface of the operating apparatus, and a plurality of trackball assemblies 210 may be provided on the second surface and/or the side surface of the operating apparatus, respectively. Alternatively, a plurality of trackball assemblies 210 and a plurality of touchpad assemblies 220 may be arranged on the plurality of surfaces of the operating apparatus, respectively. By mounting a plurality of trackball assemblies 210 and/or touchpad assemblies 220 on the plurality of surfaces of the same operating apparatus, respectively, on the one hand, it is convenient for the operator to operate (for example, flip) to select a preferred assembly (trackball assembly or touchpad assembly); and on the other hand, the provision of the plurality of assemblies is equivalent to the provision of a “spare assembly”. When an assembly on a certain operating surface fails, an assembly on another operating surface may be selected, thereby extending the service life of the entire operating apparatus and improving the user's use experience.

In some embodiments, the trackball assembly and the touchpad assembly are mounted on different operating surfaces of the operating apparatus. Even if the operating apparatus includes a plurality of trackball assemblies and/or touchpad assemblies, they are disposed on different operating surfaces of the operating apparatus.

In some embodiments, the operating apparatus is provided as a cylinder, the trackball assembly and the touchpad assembly are disposed on two opposite circular surfaces of the cylinder, respectively, and the operating apparatus can be flipped to select the trackball assembly or the touchpad assembly to connect same to the ultrasound imaging system and control the ultrasound imaging system.

In some other embodiments, the operating apparatus may also be provided as a cube, the trackball assembly and the touchpad assembly are disposed on any two surfaces of the cube, respectively, and the operating apparatus can be flipped to select the trackball assembly or the touchpad assembly to connect same to the ultrasound imaging system and control the ultrasound imaging system. Of course, a plurality of trackball assemblies and/or a plurality of touchpad assemblies can be disposed on any multiple surfaces of the cube, respectively.

Although it has been defined above that the operating apparatus may be a cylinder or a cube, it should be understood by those skilled in the art that the operating apparatus may be provided in any suitable shape, for example a cuboid or other irregular shapes.

The operating apparatus 200 further includes a first set of connectors 231 and a second set of connectors 232 on at least one surface of the plurality of operating surfaces. The first set of connectors 231 is used to connect the trackball assembly 210, and the second set of connectors 232 is used to connect the touchpad assembly 220. In some embodiments, the first set of connectors 231 and the second set of connectors 232 are mounted on other surfaces where the trackball assembly and the touchpad assembly are not disposed.

Specifically, with the operating apparatus being a cylinder as an example, when the trackball assembly 210 and the touchpad assembly 220 are disposed on two opposing surfaces (e.g., the first surface and the second surface) of the cylinder, the first set of connectors 231 and the second set of connectors 232 are disposed on the side surface of the operating apparatus.

Each set of connectors 231 and the second connectors 232 each includes a plurality of interfaces that may be used in cooperation to connect the trackball assembly 210 or touchpad assembly 220 to the ultrasound imaging system, and the ultrasound imaging system may then be controlled by the trackball assembly 210 or touchpad assembly 220. Specifically, the first set of connectors 231 includes four interfaces for connecting the trackball assembly 210 into the ultrasound imaging system, and the second set of connectors 232 also includes four interfaces for connecting the touchpad assembly 220 into the ultrasound imaging system.

The first set of connectors 231 and the second set of connectors 232 each include four interfaces, and the order of the four interfaces of the first set of connectors 231 is arranged opposite to the order of the four interfaces of the second set of connectors 232. Specifically, it is assumed that the first set of connectors 231 includes four interfaces A1 (VBUS), A2 (D−), A3 (D+) and A4 (GND) which are arranged to extend from the first surface to the second surface, and the second set of connectors 231 includes four interfaces B4 (GND), B3 (D+), B2 (D−) and B1 (VBUS) which are arranged to extend from the first surface to the second surface. Such an arrangement enables the operating apparatus to be connected to a corresponding connector after being flipped and then connected to the ultrasound imaging system. Under certain conditions, it can be determined by the connected connector whether the trackball assembly or the touchpad assembly is currently connected to the ultrasound imaging system. Of course, the number of connectors in the operating apparatus corresponds to the number of trackball assemblies and touchpad assemblies provided, and the connectors may include any number of interfaces and any set of interface functions.

To specifically determine whether the trackball assembly or the touchpad assembly is connected to the ultrasound imaging system, that is, to determine whether an operating surface on which the trackball assembly or the touchpad assembly is mounted is placed upward on the input apparatus or exposed to the input apparatus, there are a variety of methods. For example, it may be determined through different buses to which each set of connectors is connected, or when each set of connectors is connected to the processor of the ultrasound imaging system by means of a same bus, it may be determined by providing a sensor on the input apparatus or the operating apparatus. Of course, it may also be determined in any other manner.

Specifically, in some embodiments, the first set of connectors and the second set of connectors are connected to the ultrasound imaging system by means of a same bus, and the operating apparatus further includes a sensor for determining a connection state of the trackball assembly or the touchpad assembly with the ultrasound imaging system. Specifically, the connection state includes a successful connection between the trackball assembly and the ultrasound imaging system, a successful connection between the touchpad assembly and the ultrasound imaging system, and neither the trackball assembly nor the touchpad assembly being connected to the ultrasound imaging system.

Specifically, the sensor may be a Hall sensor, and a magnet is provided inside the operating apparatus near one end of the trackball assembly or the touchpad assembly. One end of the Hall sensor is mounted near the top surface (first surface) or the bottom surface (second surface) of the operating apparatus, so that it can output a high level and a low level on the basis of the Hall effect, thereby determining which assembly in the ultrasound imaging system is currently connected. Specifically, assuming that a magnet is mounted near one end of the trackball assembly and a Hall sensor is mounted near the bottom surface of the operating apparatus, when the Hall sensor outputs a high level, it represents that the touchpad assembly faces upward or is used at this time to control the ultrasound imaging system, and when the Hall sensor outputs a low level, it represents that the trackball assembly faces upward or is used at this time to control the ultrasound imaging system. Alternatively, assuming that a magnet is mounted near one end of the touchpad assembly and the Hall sensor is mounted near the bottom of the operating apparatus, when the Hall sensor outputs a high level, it represents that the trackball assembly faces upward or is used at this time, and when the Hall sensor outputs a low level, it represents that the touchpad assembly faces upward or is used at this time. By providing the sensor, on the one hand, two or more sets of connectors can be connected using a same bus, saving costs, and on the other hand, it can be determined at a lower cost whether the trackball assembly or the touchpad assembly is used to control the ultrasound imaging system.

Of course, the sensor may also be another type of sensor, for example, a gravity sensor, a gyroscope, or the like, as long as it can be determined which assembly is disposed upward.

In other embodiments, the first set of connectors and the second set of connectors are connected to the ultrasound imaging system by means of different buses, and the processor can determine the connection state of the trackball assembly or the touchpad assembly with the ultrasound imaging system on the basis of the connected bus. Specifically, when different buses are used to connect different connectors, the processor can determine whether the trackball assembly or the touchpad assembly is connected to the ultrasound imaging system on the basis of the connection state of the buses, whether there is data or signal transmission, etc. Of course, it may also be determined in any other suitable manner. For example, the user may manually input or select a corresponding option or configuration or the like after viewing the operating apparatus.

Regardless of which of the above manners is used to determine whether the trackball assembly or the touchpad assembly is connected to the ultrasound imaging system, when the processor receives an associated connection state signal, the processor can accordingly disable related functions of the other assembly that has not been successfully connected. Specifically, assuming that the processor now receives that the trackball assembly faces upward or is selected, the processor will disable all functions of the touchpad assembly at this time, and load related functions or configurations of the trackball assembly so that the user or operator's operation on the trackball assembly can be identified and controlled. Such an arrangement can prevent the occurrence of misoperation or control errors. Conversely, if the processor receives that the touchpad assembly faces upward or is selected, then the processor will disable all functions of the trackball assembly and load related functions or configurations of the touchpad assembly so that the user or operator's operation on the touchpad assembly can be identified and controlled.

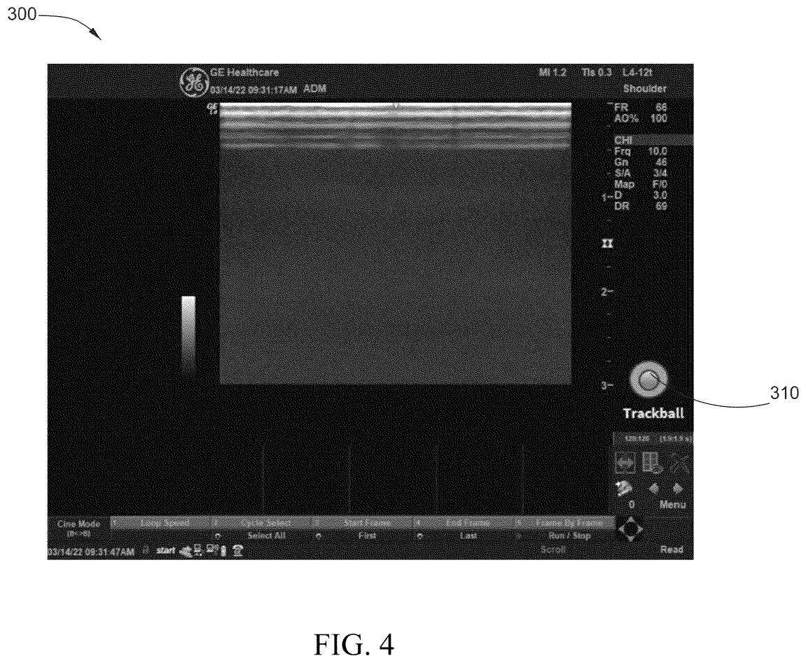

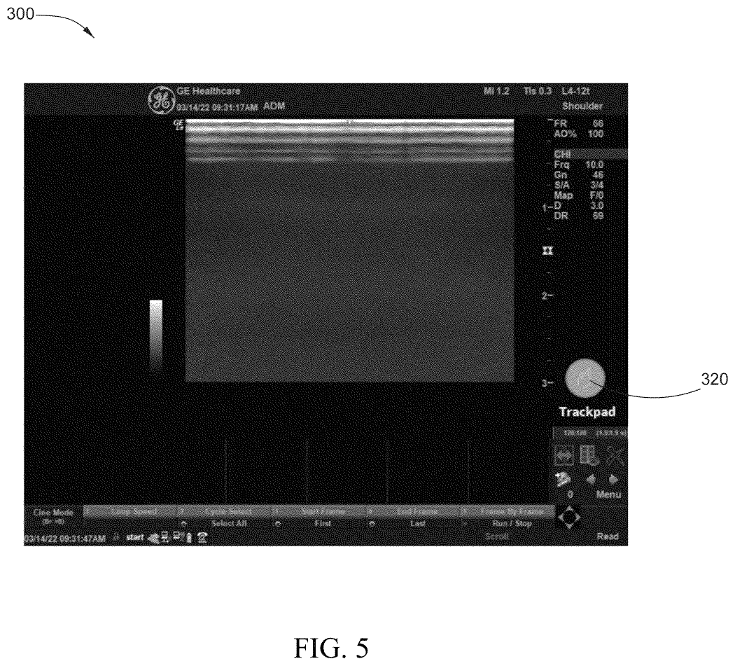

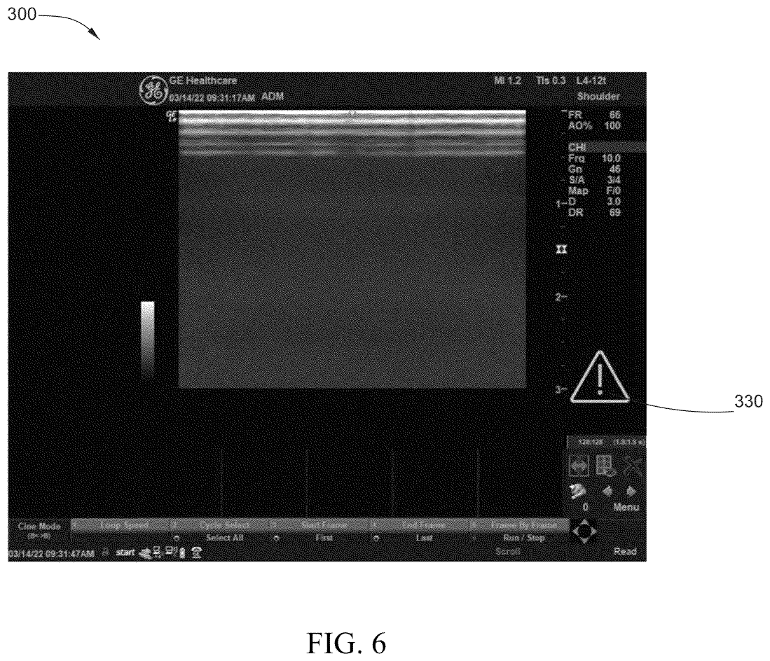

To further facilitate the user to intuitively know or learn which assembly is currently connected to the ultrasound imaging system, the display apparatus of the ultrasound imaging system may include a user interface 300 on which at least one mark can be displayed for indicating the connection state of the trackball component or the touchpad assembly. Specifically, FIGS. 4-6 show schematic views of user interfaces according to some embodiments of the present invention. As shown in FIG. 4, when the processor determines that the trackball assembly faces upward or is connected to the ultrasound imaging system, a mark 310 such as an icon or text indicating that the functions of the trackball assembly are activated is displayed on the user interface. As shown in FIG. 5, when the processor determines that the touchpad assembly faces upward or is connected to the ultrasound imaging system, a mark 320 such as an icon or text indicating that the functions of the touchpad assembly are activated is displayed on the user interface. As shown in FIG. 6, when the processor determines that neither the trackball assembly nor the touchpad assembly has been successfully connected to the ultrasound imaging system, a mark 330 such as an icon or text indicating connection failure is displayed on the user interface.

Of course, the mark indicating the connection state of the trackball assembly or the touchpad assembly is not limited to the form shown in FIGS. 4 to 6, and may be displayed visually in any other suitable manner. For example, different prompt sounds may be used for prompting, or other graphic or text labels or the like may be used.

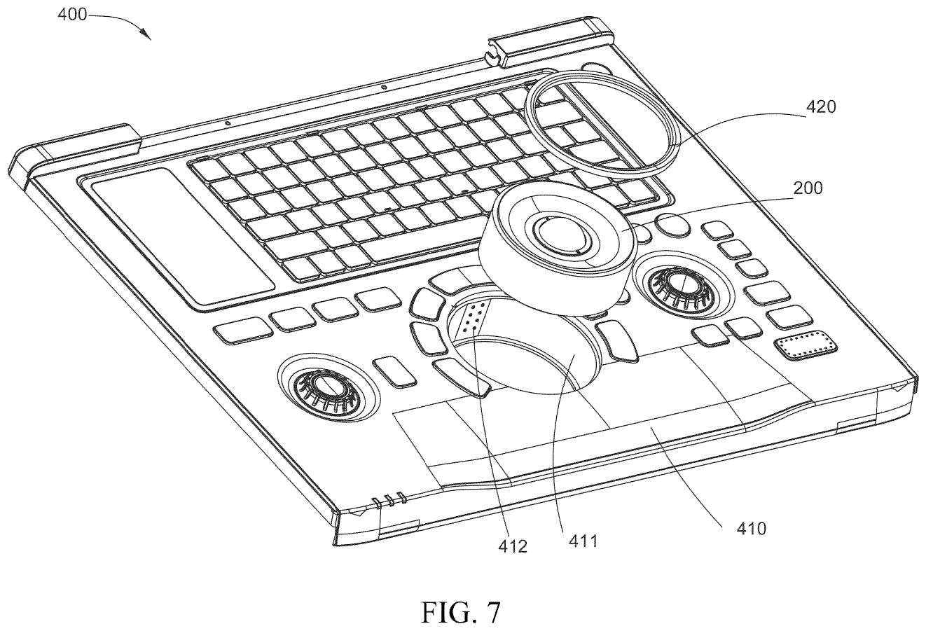

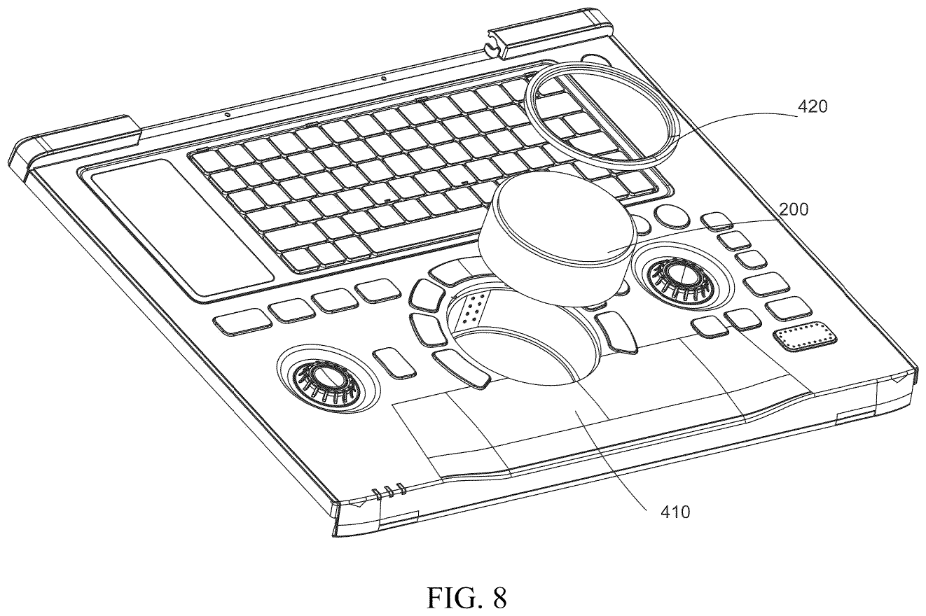

FIG. 7 shows an exploded view of an input apparatus according to some embodiments of the present invention, and FIG. 8 shows an exploded view of an input apparatus according to some other embodiments of the present invention. As shown in FIGS. 7-8, the input apparatus 400 of the ultrasound imaging system further includes an operation panel 410. The operation panel 410 includes a frame 411 that can be used to mount the operating apparatus 200. Specifically, the operation panel 410 is further provided with a plurality of operation keys for performing operations of different functions.

In some embodiments, an inner wall of the frame 411 is further provided with a connection unit 412 that matches or mates with the first set of connectors and the second set of connectors, so that when the operating apparatus 200 is placed in the frame 411, the first set of connectors and the second set of connectors are connected or contacted with the connection unit to transmit the operation on the operating apparatus to the processor of the ultrasound imaging system.

The input apparatus 400 further includes a fixing member 420. The fixing member 420 can be detachably mounted on the top of the operating apparatus 200 for fixing the operating apparatus 200 in the operation panel 410. By fixing the operating apparatus 200 in the operation panel 410 using the fixing member 420, the position of the operating apparatus can be locked, preventing the operating apparatus from being displaced during use.

In some embodiments, the fixing member 420 has a size that is approximately the same as that of (the first or second surface of) the operating apparatus. Specifically, the fixing member may have a ring shape. Of course, it may also have a rectangular shape or a square shape with a circular opening at a central portion thereof, or the like, that is, the fixing member has a shape of an outer square and an inner circle, and the shape of the fixing member is not limited.

In some embodiments, a rubber ring is provided on the side of the fixture 420 in contact with the operating apparatus to achieve a waterproof function and facilitate cleaning and disinfection.

In some embodiments, at least one protrusion is provided on the fixing member 420 to facilitate the user to operate the fixing member 420. Specifically, two protrusions may be provided on the fixing member 420. The provision of the two protrusions may facilitate the user to lift, place or rotate the fixing member.

In some embodiments, the operating apparatus 200 can be rotated with respect to the frame 411 (or the operation panel), and at least one limiting unit is provided on an inner wall of the frame 411 for limiting a limit position of the rotation of the operating apparatus 200. In the present application, the structure of the limiting unit is not particularly limited as long as it can achieve a limiting function. For example, a limiting block is mounted on the side surface of the operating apparatus, and a groove mating with the limiting block is provided on the inner wall of the frame.

In some embodiments, an indicator (e.g., an arrow) is provided on the operating apparatus 200 or the fixing member 420, and at least one position symbol (e.g., an unlocking and/or locking icon, etc.) that matches the indicator is provided on the frame to indicate an unlocking position or a fixing position of the operating apparatus with respect to the frame. Specifically, the unlocking position is a position wherein the operating apparatus can be taken out or flipped with respect to the frame, and the fixing position is a position wherein the operating apparatus can be fixed with respect to the frame. The fixing position may also be a position defined by at least one limiting unit.

Specifically, an indicator is provided on the operating apparatus or the fixing member, and only one position symbol, for example, an unlocking icon, is provided on the frame. When the indicator in the operating apparatus is aligned with the position symbol on the frame, the operating apparatus is in the unlocking position with respect to the frame, that is, at the unlocking position, the operating apparatus can be taken out or directly flipped. When the operating apparatus is placed or flipped, the operating apparatus can be rotated in a predetermined direction (for example, counterclockwise or clockwise) to a position defined by the limiting unit, that is, to the fixing position. At this time, the position of the operating apparatus is fixed.

Of course, the operating apparatus or the fixing member may be provided with one indicator, and the frame may be provided with a plurality of position symbols, for example, two position symbols, a first position symbol (unlocking position symbol) and a second position symbol (fixing position symbol). Therefore, when the indicator in the operating apparatus is aligned with the first position symbol on the frame, the operating apparatus is in the unlocking position with respect to the frame, that is, at the unlocking position, the operating apparatus can be taken out or directly flipped. After the operating apparatus is placed or flipped, the operating apparatus can be rotated in a predetermined direction (e.g., counterclockwise or clockwise) until the indicator is aligned with the second position symbol on the frame, that is, to the fixing position. At this time, the position of the operating apparatus is fixed.

In some embodiments, the operating apparatus 200 can be rotated with respect to the operation panel 410 and then taken out for flipping or directly flipped with respect to the operation panel, so as to select the trackball assembly or the touchpad assembly.

Specifically, the operating apparatus can be taken out from the frame and then manually flipped, and the assembly on the other side is flipped upward, and then the operating apparatus can be placed back into the frame. In some other embodiments, the operating apparatus can be flipped directly inside the frame so that the assembly on the other side is flipped upward. The direct flipping may be manually controlled by the user or operator. Of course, it may also be automatically controlled. Specifically, brackets may be provided at central positions of two sides of the operating apparatus, respectively, and grooves are provided at opposite positions of an inner wall of the frame. After the operating apparatus is placed into the frame, the operating apparatus directly enters the corresponding grooves, and the operating apparatus is fixed in the frame. At this time, the two brackets can form a rotation axis, and the operating apparatus can be directly flipped with respect to the rotation axis formed by the two brackets, so that the trackball assembly and the touchpad assembly can also be switched.

With the manner in which the operating apparatus is taken out and then manually flipped, as an example, assuming that the trackball assembly needs to be converted into the touchpad assembly, first, the operating apparatus and the fixing member may be rotated together from the fixing position to the unlocking position by the protrusion provided on the fixing member. Then, at the unlocking position, the fixing member and the operating apparatus are taken out of the operation panel (as shown in FIG. 7). After that, the operating apparatus is manually flipped until the touchpad assembly faces upward, and the fixing member is fixed on the upper surface of the touchpad assembly (as shown in FIG. 8). Subsequently, the fixing member and the operating apparatus are placed together into the operation panel again, and then the fixing member and the operating apparatus are rotated together to the fixing position (the position limited by the limiting unit or the position indicated by the indicator aligning with the position symbol). At this time, the touchpad assembly can be connected to the ultrasound imaging system, so as to control the ultrasound imaging system.

With the manner in which the operating apparatus can be directly flipped, as an example, first, the operating apparatus and the fixing member may be rotated together from the fixing position to the unlocking position by the protrusion provided on the fixing member. Then, at the unlocking position, the fixing member is taken out of the operation panel, and the operating apparatus is held in the frame of the operation panel. After that, the operating apparatus is directly flipped until the touchpad assembly faces upward, and then the fixing member is placed on the upper surface of the touchpad assembly again. Subsequently, the fixing member and the operating apparatus are rotated together to the fixing position. At this time, the touchpad assembly can be connected to the ultrasound imaging system, so as to control the ultrasound imaging system.

Although it is defined in the above manner that the fixing member and the operating apparatus are rotated and taken out together, it should be understood by those skilled in the art that the fixing member may be directly taken out, and then only the operating apparatus may be rotated to achieve flipping.

Some embodiments of the present invention provide an operating apparatus and an input apparatus for an ultrasound imaging system, and an ultrasound imaging system, wherein the trackball assembly and the touchpad assembly are integrated into a whole (operating apparatus), so that the user can select an assembly to be used according to his own preference to control the ultrasound imaging system, thereby improving the user's use experience. Furthermore, the trackball assembly and the touchpad assembly are mounted on different operating surfaces, so that the user can easily operate (e.g., flip) to change the related assembly. The process is simple, and it is easy to operate.

Exemplary embodiments of the present invention provide an operating apparatus for an ultrasound imaging system, the operating apparatus comprising a plurality of operating surfaces, wherein at least one of the plurality of operating surfaces is mounted with a trackball assembly and at least one other of the plurality of operating surfaces is mounted with a touchpad assembly, and the operating apparatus can be flipped so that one of the trackball assembly and the touchpad assembly can be selected and then used for controlling the ultrasound imaging system.

Specifically, the plurality of operating surfaces include a first surface and a second surface that are disposed opposite to each other, and the trackball assembly and the touchpad assembly are mounted on the first surface and the second surface, respectively.

Specifically, the operating apparatus further includes a first set of connectors and a second set of connectors mounted on at least one other of the plurality of operating surfaces, wherein the first set of connectors is used to connect the trackball assembly to the ultrasound imaging system, and the second set of connectors is used to connect the touchpad assembly to the ultrasound imaging system.

Specifically, the first set of connectors and the second set of connectors are connected to the ultrasound imaging system by means of a same bus. Specifically, the operating apparatus further includes a sensor for determining a connection state of the trackball assembly or the touchpad assembly with the ultrasound imaging system.

Specifically, the ultrasound imaging system includes a processor, the first set of connectors and the second set of connectors are connected to the ultrasound imaging system by means of different buses, and the processor can determine, on the basis of the connected buses, a connection state of the trackball assembly or the touchpad assembly with the ultrasound imaging system.

Specifically, the operating apparatus is a cylinder, the trackball assembly and the touchpad assembly are disposed on two opposite circular surfaces of the cylinder, respectively, and the operating apparatus can be flipped to select the trackball assembly or the touchpad assembly to connect same to the ultrasound imaging system and control the ultrasound imaging system.

Exemplary embodiments of the present invention further provide an input apparatus for an ultrasound imaging system, the input apparatus comprising an operating apparatus and an operation panel, wherein the operating apparatus comprises a plurality of operating surfaces, at least one of the plurality of operating surfaces is mounted with a trackball assembly and at least one other of the plurality of operating surfaces is mounted with a touchpad assembly, the operating apparatus can be flipped so that one of the trackball assembly and the touchpad assembly can be selected and then used for controlling the ultrasound imaging system, and the operation panel has a frame to which the operating apparatus can be mounted.

Specifically, the input apparatus further includes a fixing member, wherein the fixing member can be detachably mounted on a top of the operating apparatus for fixing the operating apparatus in the operation panel.

Specifically, an indicator is provided on the operating apparatus or the fixing member, and at least one position symbol matching the indicator is provided on the frame for indicating an unlocking position and a fixing position of the operating apparatus with respect to the frame.

Specifically, the operating apparatus can be rotated with respect to the frame, and the frame is provided with at least one limiting unit for limiting a limit position of rotation of the operating apparatus.

Specifically, the operating apparatus can be rotated with respect to the frame, taken out and then flipped, or directly flipped with respect to the frame, so as to select the trackball assembly or the touchpad assembly to control the ultrasound imaging system.

Exemplary embodiments of the present invention further provide an ultrasound imaging system including an input apparatus, the input apparatus including an operating apparatus and an operation panel, wherein the operating apparatus includes a plurality of operating surfaces, at least one of the plurality of operating surfaces is mounted with a trackball assembly and at least one other of the plurality of operating surfaces is mounted with a touchpad assembly, the operating apparatus can be flipped so that one of the trackball assembly and the touchpad assembly can be selected and then used for controlling the ultrasound imaging system, and the operation panel has a frame to which the operating apparatus can be mounted.

Specifically, the ultrasound imaging system further includes a user interface for displaying at least one mark for displaying a connection state of the trackball assembly or the touchpad assembly.

Some exemplary embodiments have been described above; however, it should be understood that various modifications may be made. For example, suitable results can be achieved if the described techniques are performed in a different order and/or if components in the described systems, architectures, devices, or circuits are combined in different ways and/or replaced or supplemented by additional components or equivalents thereof. Accordingly, other implementations also fall within the scope of protection of the claims.

1. An operating apparatus for an ultrasound imaging system, the operating apparatus comprising a plurality of operating surfaces, wherein at least one of the plurality of operating surfaces is mounted with a trackball assembly and at least one other of the plurality of operating surfaces is mounted with a touchpad assembly, and the operating apparatus can be flipped so that one of the trackball assembly and the touchpad assembly can be selected and then used for controlling the ultrasound imaging system.

2. The operating apparatus according to claim 1, wherein the plurality of operating surfaces comprise a first surface and a second surface that are disposed opposite to each other, and the trackball assembly and the touchpad assembly are mounted on the first surface and the second surface, respectively.

3. The operating apparatus according to claim 1, further comprising a first set of connectors and a second set of connectors mounted on at least one of the plurality of operating surfaces, wherein the first set of connectors is used to connect the trackball assembly to the ultrasound imaging system, and the second set of connectors is used to connect the touchpad assembly to the ultrasound imaging system.

4. The operating apparatus according to claim 3, wherein the first set of connectors and the second set of connectors are connected to the ultrasound imaging system by means of a same bus.

5. The operating apparatus according to claim 4, further comprising a sensor for determining a connection state of the trackball assembly or the touchpad assembly with the ultrasound imaging system.

6. The operating apparatus according to claim 3, wherein the ultrasound imaging system comprises a processor, the first set of connectors and the second set of connectors are connected to the ultrasound imaging system by means of different buses, and the processor can determine, on the basis of the connected buses, a connection state of the trackball assembly or the touchpad assembly with the ultrasound imaging system.

7. The operating apparatus according to claim 1, wherein the operating apparatus is a cylinder, the trackball assembly and the touchpad assembly are disposed on two opposite circular surfaces of the cylinder, respectively, and the operating apparatus can be flipped to select the trackball assembly or the touchpad assembly to connect same to the ultrasound imaging system and control the ultrasound imaging system.

8. An input apparatus for an ultrasound imaging system, the input apparatus comprising:
an operating apparatus comprising a plurality of operating surfaces, wherein at least one of the plurality of operating surfaces is mounted with a trackball assembly and at least one other of the plurality of operating surfaces is mounted with a touchpad assembly, and the operating apparatus can be flipped so that one of the trackball assembly and the touchpad assembly can be selected and then used for controlling the ultrasound imaging system; and
an operation panel having a frame to which the operating apparatus can be mounted.

9. The input apparatus according to claim 8, further comprising a fixing member, wherein the fixing member can be detachably mounted on a top of the operating apparatus for fixing the operating apparatus in the operation panel.

10. The input apparatus according to claim 9, wherein an indicator is provided on the operating apparatus or the fixing member, and at least one position symbol matching the indicator is provided on the frame for indicating an unlocking position and a fixing position of the operating apparatus with respect to the frame.

11. The input apparatus according to claim 8, wherein the operating apparatus can be rotated with respect to the frame, and the frame is provided with at least one limiting unit for limiting a limit position of rotation of the operating apparatus.

12. The input apparatus according to claim 8, wherein the operating apparatus can be rotated with respect to the frame, taken out and then flipped, or directly flipped with respect to the frame, so as to select the trackball assembly or the touchpad assembly to control the ultrasound imaging system.

13. An ultrasound imaging system, comprising:
A probe comprising a transducer configured to emit and receive an ultrasonic signal;
an operating apparatus comprising a plurality of operating surfaces, wherein at least one of the plurality of operating surfaces is mounted with a trackball assembly and at least one other of the plurality of operating surfaces is mounted with a touchpad assembly, and the operating apparatus can be flipped so that one of the trackball assembly and the touchpad assembly can be selected and then used for controlling the ultrasound imaging system, the operating apparatus being configured to control the transducer;
an operation panel having a frame to which the operating apparatus can be mounted.

14. The ultrasound imaging system according to claim 13, further comprising a user interface for displaying at least one mark for displaying a connection state of the trackball assembly or the touchpad assembly.

15. The operating apparatus according to claim 13, wherein the plurality of operating surfaces comprise a first surface and a second surface that are disposed opposite to each other, and the trackball assembly and the touchpad assembly are mounted on the first surface and the second surface, respectively.

16. The operating apparatus according to claim 13, further comprising a first set of connectors and a second set of connectors mounted on at least one of the plurality of operating surfaces, wherein the first set of connectors is used to connect the trackball assembly to the ultrasound imaging system, and the second set of connectors is used to connect the touchpad assembly to the ultrasound imaging system.

17. The operating apparatus according to claim 16, wherein the first set of connectors and the second set of connectors are connected to the ultrasound imaging system by means of a same bus.

18. The operating apparatus according to claim 17, further comprising a sensor for determining a connection state of the trackball assembly or the touchpad assembly with the ultrasound imaging system.

19. The operating apparatus according to claim 16, wherein the ultrasound imaging system comprises a processor, the first set of connectors and the second set of connectors are connected to the ultrasound imaging system by means of different buses, and the processor can determine, on the basis of the connected buses, a connection state of the trackball assembly or the touchpad assembly with the ultrasound imaging system.

20. The operating apparatus according to claim 1, wherein the operating apparatus is a cylinder, the trackball assembly and the touchpad assembly are disposed on two opposite circular surfaces of the cylinder, respectively, and the operating apparatus can be flipped to select the trackball assembly or the touchpad assembly to connect same to the ultrasound imaging system and control the ultrasound imaging system.
